(19) United States
(12) Patent Application Publication
(10) Pub. No.: US 2011/0160872 A1
(43) Pub. Date: Jun. 30, 2011

(54) Artificial Limb Protection System

(76) Inventor: Marinus Todd Westrate, Kalamazoo, MI (US)

(21) Appl. No.: 12/971,164

(22) Filed: Dec. 17, 2010

Related U.S. Application Data
(60) Provisional application No. 61/290,233, filed on Dec. 27, 2009.

(51) Int. Cl. A61F 2/78

(52) U.S. Cl. 623/27

(57) ABSTRACT

Disclosed herein is an artificial limb protection system comprising at least one sheath and optionally, a sealing means. The sealing means may be worn over a jointed body part such as a shoulder or a hip. The sheath may be stretched over a prosthesis and over a substantial portion of the amputation stump. The lower portion of the sealing means is folded over the top portion of the sheath to prevent falling water from coming in contact with the prosthetic.

### RELATED U.S. APPLICATION DATA

This application is a non-provisional of and claims the benefit of the filing date of U.S. Provisional Application No. 61/290,233 filed on 27 Dec. 2009.

### BACKGROUND

The present invention relates generally to the field of prosthetics, and in particular to a protective shield for a prosthetic limb. Many individuals with a limb amputation have a prosthesis that functions to replace the lost limb and assist them in accomplishing everyday tasks. These prosthetics are useful in almost any situation; however they are unable to get wet. Several devices are known that function to protect a prosthetic limb when it is worn by the user as he or she engages in water related activities. In particular, covers for a prosthetic limb are used for protection of the artificial limb itself and for decorative purposes. Among these are devices comprising a one-piece hollow sealing means that serves the purpose of a sleeve for the prosthetic limb. One such device comprises a sleeve and a plurality of cinching members designed to create a seal. Another is in the form of a bag that is capable of being shortened or lengthened depending on the size of the prosthetic. Advantageously, all of these devices provide some level of waterproofing that allow the user to engage in water related activities that would otherwise not be possible without risking serious damage to a prosthetic limb. However, these measures are not fully effective.

### BRIEF SUMMARY OF THE INVENTION

The known prosthetic protectors do not provide sufficient means for a water tight seal around a prosthetic limb. Amputees who wish to engage in water related activities, even something as simple as bathing, must remove the prosthetic, thereby making the activity extremely difficult if not dangerous or even impossible to accomplish. If the amputee wishes to wear his or her prosthetic, she or he must choose between risking damage from water leaking around a seal or withstand uncomfortable (and often ineffective) cinching around the amputation stump. The present invention ensures that a prosthetic limb stays dry while bathing, boating, or participating in any other water-related activity. It is also portable to ensure that it is readily available when needed and avoids problems of leaking and discomfort.

In a protection system for a prosthetic embodying the principles of the invention, the problem of leaking and/or discomfort is solved by a two-piece system. Such a system avoids the uncomfortable cinching method as well as an ineffective seal. In this way, water does not have the opportunity to come into contact with the prosthetic limb and damage it, all while still permitting the user to engage in water related activities without having to remove his or her prosthesis.

The two-piece system can be configured to work with any type, size, or number of prostheses. However, use with a prosthetic arm or leg have proven to be the most advantageous. Further, the components of the protection system are designed to be durable, lightweight, portable, and inexpensive to manufacture.

### BRIEF DESCRIPTION OF THE DRAWINGS

FIG. 1 illustrates an artificial limb protection system embodying the principles of the present invention.

FIG. 2, consisting of FIGS. 2A and 2B, illustrates an embodiment of the present invention according to FIG. 1.

FIG. 3, consisting of FIGS. 3A and 3B, illustrates an embodiment of the present invention according to FIG. 1.

FIG. 4 illustrates a kit for an artificial limb protection system embodying the principles of the present invention.

FIG. 5 illustrates a method of using an artificial limb protection system embodying the principles of the present invention.

### DETAILED DESCRIPTION

Referring now to the drawings in detail, this invention will be described by way of example and with reference to preferred embodiments thereof, however, it is to be understood that modifications and improvements may be made without departing from the scope or spirit of the invention. It should be noted that various changes and modifications to the presently preferred embodiments described herein will be apparent to those skilled in the art. Such changes and modifications may be made without diminishing its attendant advantages. It is therefore intended that such changes and modifications be included within the present invention.

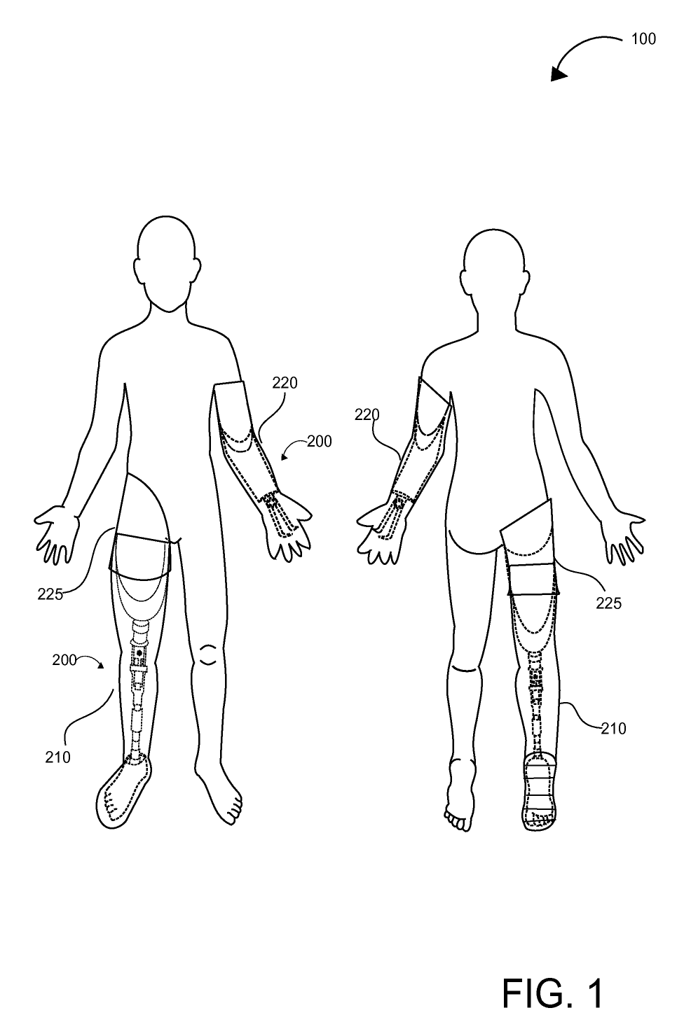

Referring now to FIG. 1, which illustrates artificial limb protection system 100 according to an embodiment of the present invention. Artificial limb protection system 100 is shown having at least one sheath 200 and at least one sealing means 225. The proximal end of sheath 200 may be substantially extended over a prosthetic and enveloped by sealing means 225 as shown. In this manner, sheath 200 and sealing means 225 may serve the purpose of preventing water and other liquids from coming in contact with a prosthetic limb. In other embodiments, sheath 200 may operate independent of sealing means 225 to create a seal against the user's amputation stump to serve the purpose of preventing water and other liquids from coming in contact with the user's prosthetic as shown.

The components of artificial limb protection system 100 may be easily put on and/or removed for portability and ease of transport. Further, the components of artificial limb protection system 100 may comprise any material sufficient to prevent liquids from penetrating the system including, but not limited to, elastomeric polymers, natural and synthetic rubbers, latex, neoprene, plastics, treated fabrics, silicone, or any other pliant materials that are suitably flexible and waterproof. The dimensions of the components of artificial limb protection system 100 are such that they are compatible with average human proportions and are designed to stretch over a standard prosthetic to create a snug fit over, for example, a prosthetic arm or leg.

It is contemplated within the present invention to protect prostheses of varying sizes and varying types, including, but not limited to, an amputation below the knee(s) 210 or above the elbow(s) 220. It should further be understood that in alternative embodiments, artificial limb protection system may comprise a user determined number of sheath(s) 200 and/or sealing means 225. By way of example, FIG. 1 depicts artificial limb protection system 100 comprising one (1) sheath 200 configured to stretch over an artificial leg above the knee 210; one (1) sealing means 225 configured to be worn over the hip; and one (1) sheath 200 configured stretch over an artificial arm above the elbow 220.

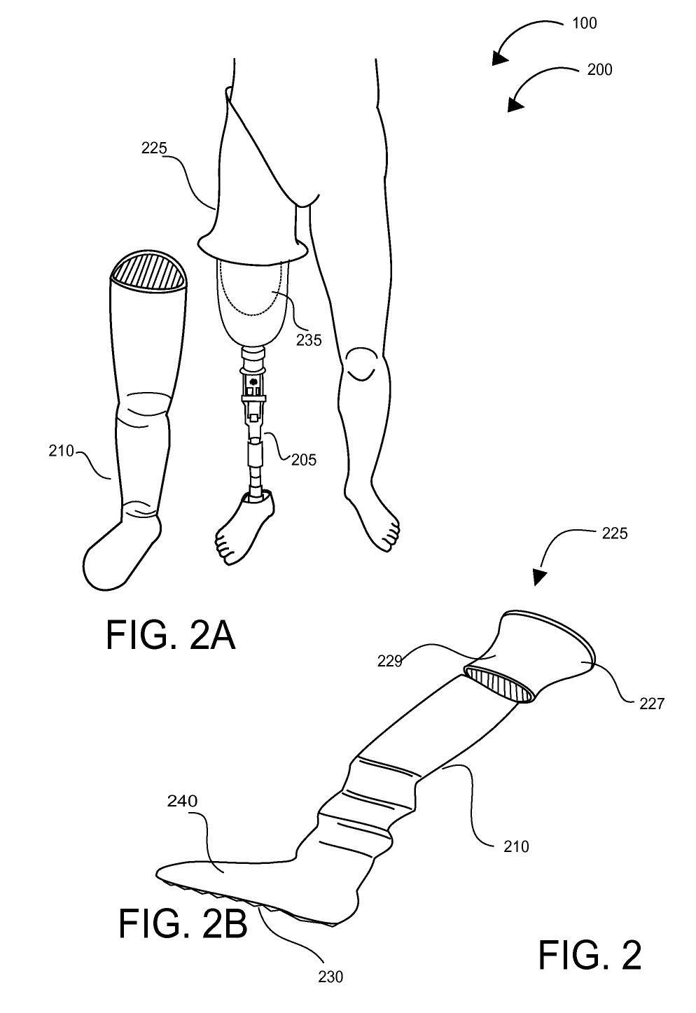

Referring now to FIG. 2, consisting of FIGS. 2A and 2B, and illustrating an embodiment of the present invention according to FIG. 1. Within the embodiment as depicted in FIG. 2A, artificial limb protection system 100 comprises one sheath 200 and one sealing means 225. Within the present embodiment, sheath 200 and sealing means 225 are configured to protect a prosthetic leg 205 above the knee 210. It is contemplated within the current invention for the dimensions of sheath 200 and sealing means 225 to conform to the dimension of any size or type or size of leg prosthesis. By way of example only, FIG. 2 depicts sheath 200 configured a prosthetic leg above the knee 210 and sealing means 225 configured to be worn over the hip and extend downward over amputation stump 235 as shown. In alternative embodiments (not shown), sealing means 225 may be configured to be worn, for example, below the hip, such as above the knee or above the ankle. Further, in alternative embodiments (not shown) sheath 200 may be configured to stretch, for example, below the knee or above the ankle.

Within the present embodiment, and as depicted in FIG. 2B, sealing means 225 may consist of a hollow, substantially cylindrical configuration as shown. Sealing means 225 further comprises upper portion 227 and lower portion 229. Lower portion 229 may consist of a smaller diameter than upper portion 227. In this manner, upper portion 227 is capable of being stretched over the widest portion of amputation stump 235 and over a jointed portion of the body, such as a hip, shoulder, ankle or knee. Lower portion 229 may then be folded up over itself creating a space between amputation stump 235 and sealing means 225 as shown. The user may then attach his or her prosthesis as usual.

Within the present embodiment, sheath 200 may be substantially hollow and cylindrical as shown and may comprise top 240 and foot 240. Sheath 200 may be a stretched over an amputation stump 235 and prosthetic leg 205. In this manner, when sheath 200 is fully stretched over prosthetic leg 205 and amputation stump 235, top 240 rests against amputation stump 235 and substantially under sealing means 225. The user may then fold lower portion 229 down such that it substantially overlaps sheath 200. In this manner, a watertight protective envelope is created over prosthetic leg 205. Foot 240 may further comprise a plurality of grip(s) 230 as shown. In this manner, grip 230 provides a rough surface for added safety to prevent the user from slipping on a slick surface. It should be understood that sheath 200 may be configured to cover any prosthesis, and is not limited to the size and shape of a prosthetic limb for an amputation stump above the knee as shown; artificial limb protection system 100 is designed to accommodate any prosthesis with any amputation stump.

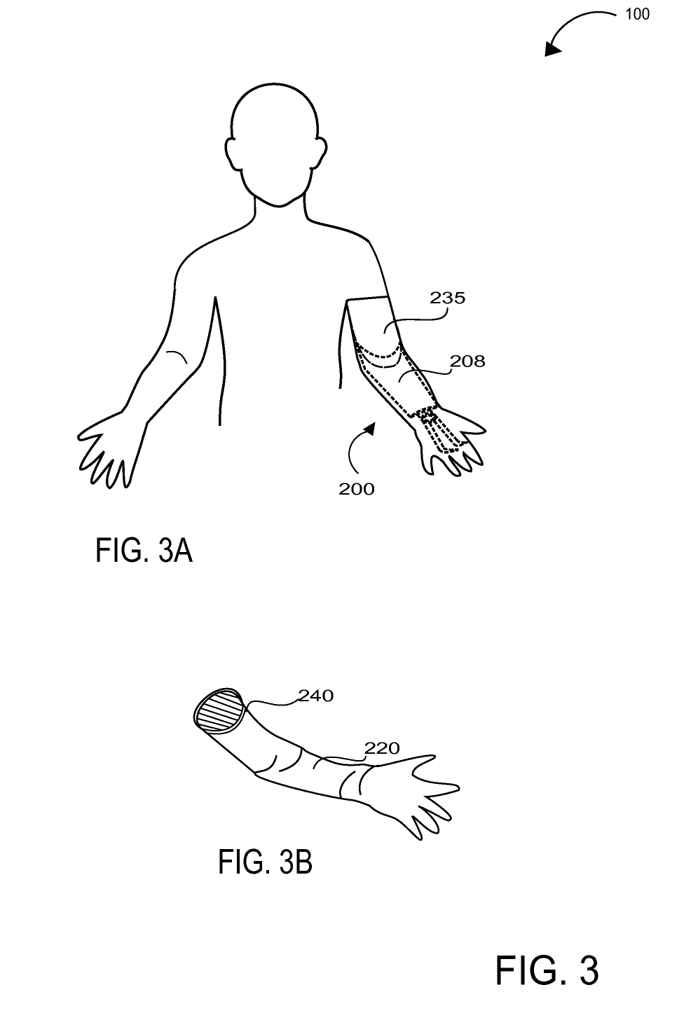

FIG. 3, consisting of FIGS. 3A and 3B, illustrates an alternative embodiment of artificial limb protection system 100 wherein sheath 200 is configured to protect a prosthetic arm 208 and amputation stump 235 below the elbow 220. By way of example. FIG. 3A illustrates artificial protection system 100 comprising one sheath 200 configured to fit over prosthetic arm 208. Within the present embodiment, and as depicted by FIG. 3B, sheath 200 may be substantially hollow and cylindrical as shown and may comprise top 240. The user attaches his or her prosthetic arm 208 as usual. Within the present embodiment, sheath 200 may then be a stretched over prosthetic arm 220 and onto an amputation stump as shown. In this manner, when sheath 200 is fully stretched over prosthetic arm 208 and amputation stump 235, top 240 rests against amputation stump 235 creating a snug watertight envelope over prosthetic arm 220. In an alternative embodiment (not shown) sealing means 225 may be used. In this manner, sealing means 225 may be configured to be worn over the shoulder, elbow, or wrist and work in conjunction with sheath 200 configured to protect a prosthetic arm or hand.

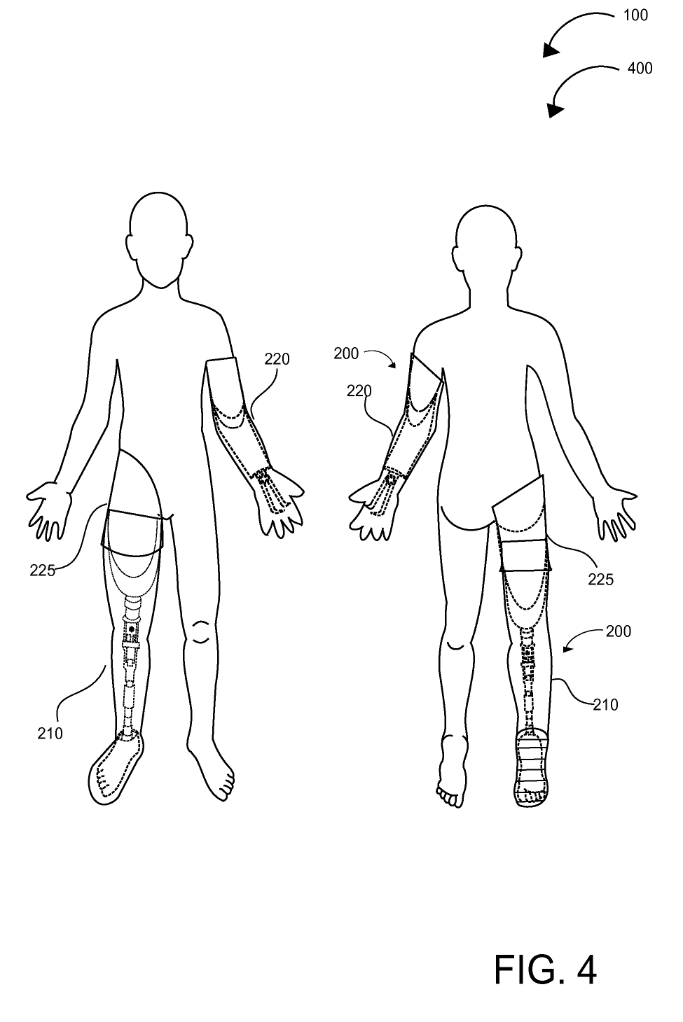

Referring now to FIG. 4, illustrating kit 400 of artificial limb protection system 100 as shown in FIGS. 1-3. Kit 400 may include at least sheath 200; at least one sealing means 225; and at least one set of user instructions. Artificial limb protection system 100 may be preferably manufactured and provided for sale in a wide variety of sizes and shapes for a wide assortment of applications. Sheath 200 may be removably put over a prosthetic, whereby top 240 may substantially cover amputation stump 235. Sealing means 225 may optionally be worn over a jointed body part such as a shoulder or a hip. Sealing means 225 may further stretch over sheath 200. Kit 400 contents may comprise the various components made from different materials as mentioned above. By way of example, kit 400 as depicted in FIG. 4 shows one (1) sheath 200 configured to cover prosthetic arm below the elbow 220; one (1) sheath 200 configured to cover prosthetic leg above the knee 210; and one (1) sealing means 225 covering sheath 200.

The embodiments of the invention described herein are exemplary and numerous modifications, variations, and rearrangements can be readily envisioned to achieve substantially equivalent results, all of which are intended to be embraced within the spirit and scope of the invention. Finally, artificial limb protection system 100 provides a system whereby the components may be manufactured separately providing for decreased costs related to shipping. Further, the user has the additional benefit that the system is easily assemblable and disassemblable for transport between locations and in/or out of the sanitation machine. The user also benefits in that they may select individual components instead of buying a pre-packaged unit.

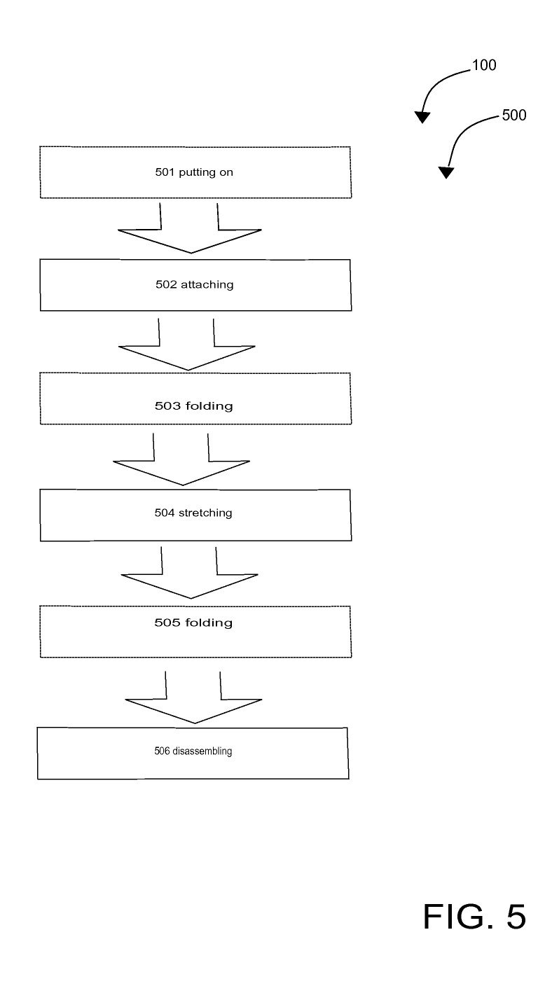

Referring now to FIG. 5, illustrating method of use 500, which may comprise the following steps of assembling and using artificial limb protection system 100 according to user preference including: step one 501 putting on sealing means 225; step two 502 attaching a prosthesis; step three 503 folding lower portion 229 up; step four 504 stretching sheath 200 over the prosthesis; and step five 505 folding lower portion 229 over top 240 of sheath. Method of use 500 further comprises optional step six 506 of disassembling and removing artificial limb protection system 100 for storage and/or transport. It should further be noted that steps 501, 503, and 505 are optional and are represented by dotted lines.

It should be noted that the steps described in the method of use can be carried out in many different orders according to user preference. Upon reading this specification, it should be noted that, under appropriate circumstances, when considering such issues as user, design, and marketing preferences, cost considerations, structural requirements, available materials, technological advances, etc., other kit contents such as, for example, including more or less components, customized parts, different color combinations, or separately sold parts may present a suitable alternative.

From the foregoing description, it should be appreciated that a preferred embodiment and a method for protecting an artificial limb are provided and present significant benefits that would be apparent to one skilled in the art. Furthermore, it should be appreciated that a vast number of variations in the embodiments exist. Lastly, it should be appreciated that these embodiments are preferred exemplary embodiments only, and are not intended to limit the scope, applicability, or configuration of the invention in any way. Rather, the foregoing detailed description provides those skilled in the art with a convenient framework for implementing a preferred exemplary embodiment of the invention. It being understood that various changes may be made in the function and arrangement of elements described in the exemplary preferred embodiment without departing from the spirit and scope of the invention as set forth in the appended claims.

What is claimed is:

1. A prosthesis protector comprising:
at least one sheath for covering said prosthesis; and
a sealer for covering sheath;

2-6. (canceled)

7. A method for protecting a prosthetic limb, said method comprising the steps of:
putting on a sealer;
folding up the lower portion of said sealer;
attaching a prosthetic limb;
stretching a sheath over said prosthetic limb; and
folding said lower portion of said sealer down over said sheath.
